(19) United States
(12) Patent Application Publication
(10) Pub. No.: US 2012/0083727 A1
(43) Pub. Date: Apr. 5, 2012

(54) DRUG DELIVERY BLADE AND METHODS FOR DELIVERING A DRUG DEPOT TO A TARGET SITE

(76) Inventor: PETER J. BARNETT, Eden Prairie, MN (US)

(21) Appl. No.: 13/250,203

(22) Filed: Sep. 30, 2011

Related U.S. Application Data
(60) Provisional application No. 61/388,254, filed on Sep. 30, 2010.

(51) Int. Cl. A61F 9/00; A61M 37/00

(52) U.S. Cl. 604/22

(57) ABSTRACT

A drug delivery blade for delivering a drug depot to a treatment site in a patient is disclosed. The drug delivery blade includes a shaft defining a longitudinal axis of the blade, a lumen that extends longitudinally from a proximal end of the shaft to a distal end of the shaft, a first beveled cut that forms a sharpened tip at the distal end of the shaft, and a flared portion near the distal end of the shaft. The flared portion of the blade includes two radially-extending blade segments having sharpened leading edges that allow the drug delivery blade to cut tissue when the blade is advanced toward a treatment site. Methods of using the drug delivery blade are also disclosed.

### CROSS-REFERENCE TO RELATED APPLICATIONS

This application claims benefit of and priority to U.S. Provisional Patent Application No. 61/388,254, filed on Sep. 30, 2010, which is fully incorporated herein by reference and made a part hereof.

### FIELD

The present invention relates to a drug delivery blade for use in delivering drug depots to target sites of a patient (e.g., an eye). Specifically, the drug delivery blade can be used to deliver drug depots (i.e., implants, microparticles, and/or gels containing drugs and/or bioactive agents) into various locations within the eye, for example, the vitreous.

### BACKGROUND

A primary difficulty in treating diseases of the eye is the inability to introduce drugs or therapeutic agents into the eye and maintain these drugs or agents at a therapeutically effective concentration in the eye for the necessary duration. Systemic administration may not be an ideal solution because, often, unacceptably high levels of systemic dosing are needed to achieve effective intraocular concentrations, with the increased incidence of unacceptable side effects of the drugs. Simple ocular instillation or application is not an acceptable alternative in many cases because the drug may be quickly washed out by tear-action or is otherwise depleted from within the eye into the general circulation. Such methods make it difficult to maintain therapeutic levels of drug for adequate time periods.

One method of maintaining a therapeutically effective concentration in the eye is by the use of sustained release drug depots (implants, microparticles, and/or gels that contain one or more drugs and/or bioactive agents). Drug depots typically include one or more drugs and/or bioactive agents that are dispersed or distributed throughout a matrix material, for example, a polymer. Drug depots may be placed in the eye in order to deliver an effective concentration of the drug and/or bioactive agent over an extended period of time. Various sites exist in the eye for implantation of a drug depot, such as the vitreous, anterior or posterior chambers of the eye, or other tissues of the eye. To avoid having to suture the incision, drug depots are ideally implanted using self-sealing methods. The use of very small needles to deliver drug depots through self-sealing incisions, however, effectively limits the amount of drug and/or bioactive agent that can be delivered because the size (e.g., diameter) of the drug depot is limited to the size of the lumen in the delivery needle.

In view of the foregoing, what is needed is a device for delivering a drug depot through a self-sealing incision where the device delivers a drug depot that has a large cross-sectional area relative to the size of the incision thereby allowing a high drug and/or bioactive agent load to be delivered by the drug depot at the treatment site.

### SUMMARY

In one embodiment, the invention relates to a drug delivery blade that is suitable for delivering a drug depot to a treatment site in a patient (e.g., an eye) with a self-sealing incision. In many embodiments, the drug delivery blade comprises: a shaft defining a longitudinal axis of the blade; a lumen that extends longitudinally from a proximal end of the shaft to a distal end of the shaft; wherein the lumen terminates at the distal end to form an opening; a first beveled cut that forms a sharpened tip at the distal end of the shaft; a flared (i.e., arrowhead-shaped) portion near the distal end of the shaft, wherein the flared portion comprises two radially-extending blade segments; and wherein the radially-extending blade segments contain sharpened leading edges at the distal end of the shaft. The radially-extending blade segments allow the drug delivery blade to cut tissue when the blade is advanced toward a treatment site.

In exemplary embodiments, the drug delivery blade of the invention is configured to cut tissue as the distal end of the blade is advanced against tissue and to not cut additional tissue as the blade is withdrawn from the cut tissue. This is accomplished by configuring the drug delivery blade with a flared portion having a sharpened distal end for cutting tissue when the blade is advanced and rounded trailing edges of the flared portion that do not cut tissue when the blade is withdrawn.

In order to increase the amount of drug that can be delivered by the drug delivery blade, in many embodiments the drug delivery blade contains a lumen that is non-circular in cross-sectional shape. In this way, a drug depot having an increased cross-sectional area can be held in the lumen and delivered by the blade using a self-sealing incision. In some embodiments, the amount of drug that can be delivered is increased by about 20% or greater, or about 30% or greater, or about 40% or greater as compared to a lumen having a circular cross-sectional shape.

In some embodiments, the drug delivery blade of the invention is sized and shaped so that it creates an incision that is self-sealing. For example, the drug delivery blade may have a width corresponding to 23 gauge (about 0.57 mm) or smaller as measured across the radially-extending blade portions and a height of 25 gauge (about 0.46 mm) or smaller in dimension as measured at 90° to the radially-extending blade portions (90° from the width of the delivery blade).

In use, the drug delivery blade of the invention is fitted to a blade holder that secures the drug delivery blade and allows it to be manipulated by the surgeon. In some embodiments, the blade holder includes a plunger or stop that is sized to fit into the lumen of the drug delivery blade. In some embodiments, the plunger slides longitudinally through the lumen to eject the drug depot. In other embodiments, the blade holder includes a retracting outer blade with a stationary stop for the drug depot.

The blade holder optionally includes a plunger and mechanism for advancing a plunger longitudinally through the lumen from a first position to a second position. In the first position the plunger is positioned inside of the lumen in a retracted position so that the distal end of the lumen can hold a drug depot. In the second position the plunger is advanced forward longitudinally down the lumen thereby ejecting the drug depot from the distal end of the drug delivery blade. Various mechanisms for advancing and retracting the plunger may be used.

In another embodiment, the invention provides a drug delivery system comprising: (a) a drug delivery blade of the invention; (b) a blade holder; and (c) a drug depot. To use the system, the drug delivery blade is attached to the blade holder so that the plunger of the blade holder slides longitudinally through the lumen of the drug delivery blade. A drug depot resides within the lumen and can be implanted at a target site (e.g., eye) by advancing the plunger so that the plunger causes the drug depot to be ejected from the opening at the distal end of the blade holder.

In yet another embodiment, the invention provides a method of administering a drug to a target site in a patient, the method comprising the steps of: providing a drug delivery system of the invention; creating an incision at the target site in the patient using the drug delivery blade; and implanting the drug depot at the target site using the blade holder to eject the drug depot from the opening of the lumen of the drug delivery blade. Examples of target sites include the eye of a patient, for example, the vitreous. In a preferred embodiment, the incision made by the drug delivery blade is a self-sealing incision.

### BRIEF DESCRIPTION OF THE FIGURES

FIG. 1 is a perspective view of the distal end of an exemplary drug delivery blade as described herein.

FIG. 2 is a distal end view of an exemplary drug delivery blade as described herein.

FIG. 3 is a cross-sectional view of the drug delivery blade of FIG. 1 taken at line 3-3.

FIG. 4 is perspective view of the proximal end of an exemplary drug delivery blade taken from the underside of the blade, as described herein.

FIG. 5 is a close-up perspective view of the distal end of an exemplary drug delivery blade as described herein.

FIG. 6 is a side view of an exemplary blade holder equipped with a drug delivery blade as described herein.

FIG. 7 is a side view of an exemplary blade holder equipped with a drug delivery blade as described herein.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular embodiments only and is not intended to be limiting.

The following description of the invention is provided as an enabling teaching of the invention in its best, currently known embodiment. To this end, those skilled in the relevant art will recognize and appreciate that many changes can be made to the various embodiments of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a leading edge” can include two or more such leading edges unless the context indicates otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

As used herein “drug depot” refers to implants, microparticles, and/or gels that can be implanted into any number of locations in patient (e.g., the eye) and which are designed such that a controlled amount of drug and/or bioactive agent can be released over time. Such drug depots, which can be solid or semi-solid, are biocompatible, and may be formed using polymers or other lower molecular weight materials. Solid drug depots useful in the invention typically have non-circular cross-sectional shape for delivery through lumens having corresponding cross-sectional shape.

As used herein “self-sealing” refers to methods of introducing drug depots through a drug delivery blade and into desired locations of a patient's eye without the need for a suture, or other like closure means, at the incision site. Such “self sealing” methods do not require that the incision site completely seal immediately upon withdrawal of the delivery device, but rather that any initial leakage is minimal and dissipates in short order such that a surgeon would not suture or otherwise provide other like closure means to the incision site.

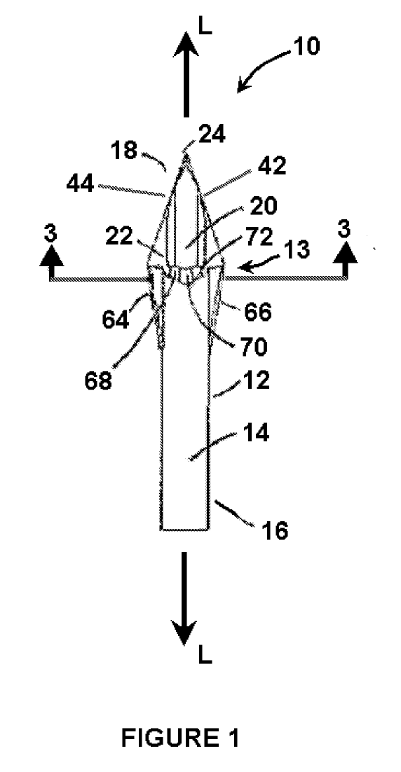

Referring to FIG. 1 an embodiment of a drug delivery blade 10 of the invention is shown. Drug delivery blade 10 includes shaft 12 that defines the longitudinal axis L of the drug delivery blade 10. Shaft 12 includes a central lumen 14 that extends from the proximal end 16 of the shaft 12 to the distal end 18 of shaft 12. At the distal end 18 of shaft 12, the central lumen 14 terminates at opening 20. The distal end 18 of shaft 12 can contain a first beveled cut 22 that forms a sharp tip 24 near opening 20.

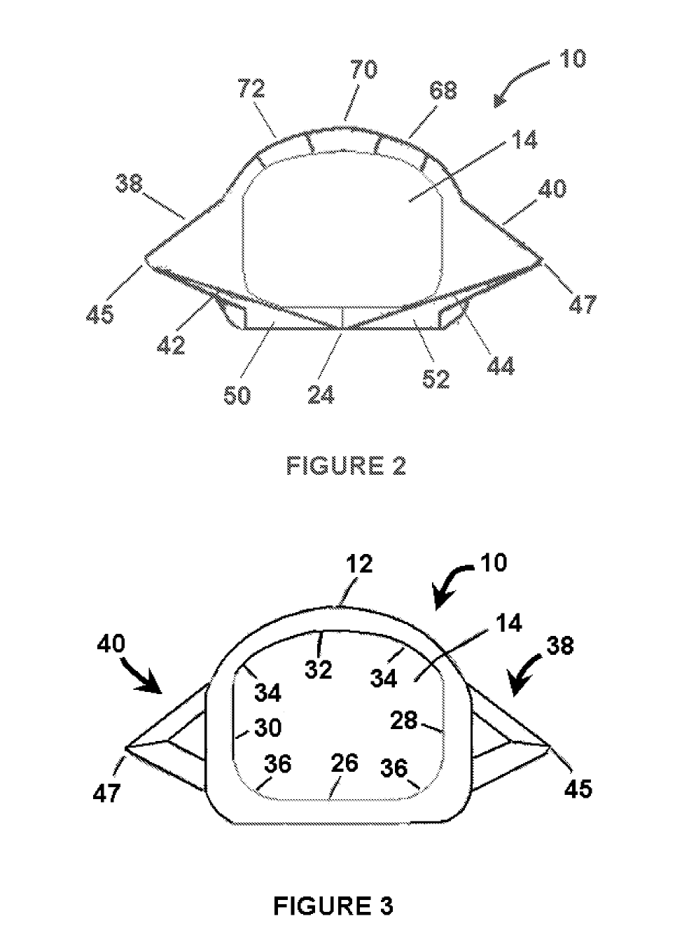

Referring now to FIGS. 1-3 and 5, drug delivery blade 10 can contain a flared or widened portion 13 near the distal end 18 of shaft 12. The flared portion 13 gives the drug delivery blade an arrowhead-like shape near the distal end 18. At the flared portion 13, the radius of the shaft increases on opposite sides of lumen 14. In an exemplary embodiment, the flared portion 13 can be made up of two blade segments 38 and 40 that extend outwardly in a radial direction from the lumen 14 of shaft 12. In the embodiment of FIG. 2, the blade segments 38 and 40 are positioned at 180° relative to each another as measured around the longitudinal axis of the shaft 12. However, it is contemplated that the blade segments 38 and 40 can be positioned relative to one another at an angle ranging from about 120 degrees to about 240 degrees. Radially extending blade segments 38 and 40 can contain sharpened leading edges 42 and 44 that extend from the tip 24 of drug delivery blade 10 to the apexes 45 and 47 of the radially extending blade portions 38 and 40. It is contemplated that the radially extending blade portions 38 and 40 can together form a cutting surface at the distal end of the delivery blade 10 for cutting tissue when the distal end of drug delivery blade 10 is advanced against tissue (e.g., eye tissue).

Referring now to FIG. 3, a cross-sectional view of drug delivery blade 10 is shown taken from the distal end 18 of blade 10. As shown in FIG. 2, central lumen 14 is non-circular in cross-sectional shape. The shape may be oval, a square with rounded corners, and the like. In some embodiments, one or more sides can be be arcuate. In the embodiment of FIG. 3, central lumen 14 has flat bottom surface 26, flat sidewall surfaces 28 and 30, and arcuate top surface 32. The top and bottom surfaces 32 and 26 meet the sidewall surfaces with rounded corners 34 and 36. It is contemplated that, in this way, the lumen 14 can have a generally smooth interior surface without angular (e.g., square) corners. The central lumen 14 can have a non-circular shape in order to increase the cross-sectional area of the lumen with respect to the size of the shaft. It is contemplated that this shape can allow the drug delivery blade to hold a larger drug depot than if a circular lumen were used. It is further contemplated that a larger drug depot can be advantageous since it can contain a larger quantity of drug or bioactive agent for delivery to the eye. The cross-sectional shape and size of the lumen may be chosen so that the cross-sectional area of the lumen 14 substantially corresponds to the cross-sectional area of the drug depot, with enough tolerance such that the drug depot can be received into and subsequently ejected from the lumen 14. In many embodiments, the cross-sectional area of lumen 14 can range from about 0.1 mm² to about 0.4 mm². In other embodiments, the cross-section area of lumen 14 can range from about 0.1 mm² to 0.2 mm². In one exemplary embodiment, the cross-sectional area of lumen 14 can be about 0.137 mm². It is contemplated that, relative to the outer dimensions of the device, the large cross-sectional area of the lumen can allow the device to hold drug depots having a relatively larger cross-sectional area while still providing a self-sealing wound. It is further contemplated that the relatively larger cross-sectional area of the drug depots allows the drug delivery blade 10 to deliver a higher loading of drug or bioactive agent to the eye.

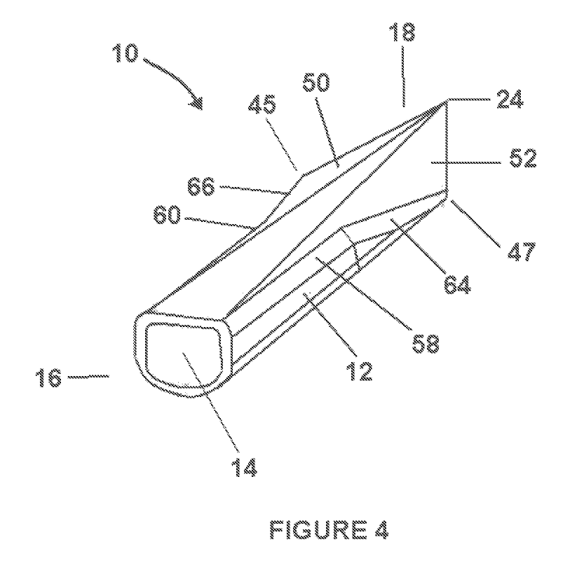

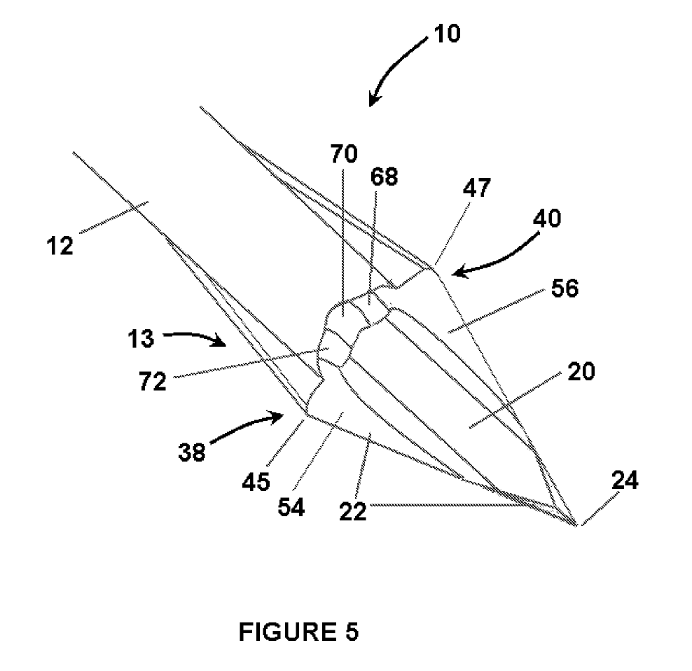

Referring now to FIG. 4, a perspective view of drug delivery blade 10 taken from the proximal end 16 of the underside of the delivery blade 10 (i.e., the side opposite opening 20) is shown. As shown in FIG. 3, drug delivery blade 10 can include longitudinal bevel cuts 50 and 52 along the bottom surface of the drug delivery blade 10. In exemplary embodiments, the longitudinal bevel cuts 50 and 52 meet with surfaces 54 and 56 formed by bevel cut 22 to form sharpened leading edges 42 and 44 of blade 10 (see, FIG. 1).

With reference to FIG. 1, it is contemplated that the angle defined by the longitudinal axis L of the blade 10 and each respective leading edge 42, 44 (measured from the point of the sharp tip at which each respective leading edge intersects with the longitudinal axis, as if the longitudinal axis and the leadings edges are in a common horizontal plane) can be any angle, including, for example, an angle ranging from about 15 degrees to about 45 degrees. With reference to FIGS. 1, 2, and 5, it is contemplated that the angle at which the surfaces 54 and 56 of the first bevel cut 22 are upwardly sloped and/or angled relative to the longitudinal axis L (measured from the point of the sharp tip at which each leading edge 42, 44 intersects with the longitudinal axis, as if the longitudinal axis and the intersection point of the sharp tip are within a common transverse plane extending upwardly through the blade 10) can be any angle, including, for example, an angle ranging from about 15 degrees to about 45 degrees.

In many embodiments, the drug delivery blade 10 of the invention can be configured to cut tissue as the blade 10 is inserted into the tissue (e.g., an eye) but not cut tissue as the blade 10 is withdrawn. In an exemplary embodiment, this can be accomplished by equipping the blade 10 with sharpened leading edges 42 and 44 for cutting as the blade is advanced through tissue and rounded trailing edges 64 and 66 so that the trailing edges do not cut tissue as the blade is withdrawn from the tissue. It is contemplated that this design can minimize damage to the eye that may occur when the blade is withdrawn from the eye after implantation of a drug depot.

Referring now to FIG. 4, a perspective view of the bottom of drug delivery blade 10 taken from the proximal end on the underside of the delivery blade 10 is shown. As shown in FIG. 4, the drug delivery blade 10 of the embodiment of FIG. 4 can include longitudinal radius cuts 58 and 60 which act to round the corners along the shaft of the device in order to form rounded trailing edges 64 and 66.

In exemplary embodiments, and referring again to FIGS. 1-2, it is contemplated that the drug delivery blade 10 can include one or more fillet cuts 68, 70, and 72. The fillet cuts can be configured to round out sharp edges that may be present due to the surface cuts, such as bevel cut 22. It is contemplated that the fillet cuts 68, 70, 72 can be further configured to contact and cut tissue as the drug delivery blade is inserted into the tissue of a patient.

The width of the drug delivery blade of the invention at its widest point (i.e., measured side-to-side across the flared portion of the blade from apex 45 to apex 47) can range from about 0.5 mm to about 1 mm. In an exemplary embodiment, the width of the drug delivery blade can range from about 0.5 mm to about 0.8 mm. The height of the drug delivery blade (i.e., measured top-to-bottom at 90° from the apex of the blade portions) can range from about 0.7 mm to about 0.3 mm. In an exemplary embodiment, the height of the drug deliver blade can range from about 0.6 mm to about 0.4 mm. In many embodiments, the sidewalls 28 and 30 of the drug delivery blade can have a thickness ranging from about 50 μm to about 100 μm. It is contemplated that the thickness of the sidewalls 28 and 30 can be substantially consistent around central lumen 14. Alternatively, the thickness of the sidewalls 28 and 30 can vary around central lumen 14. In an exemplary embodiment, the width of the drug delivery blade 10 can be 23 gauge (i.e., about 0.573 mm or less); the height of the drug delivery blade can be 25 gauge (i.e., about 0.455 mm or less), and the thickness of the sidewalls 28 and 30 can range from about 50 μm to 60 μm.

It is contemplated that the drug delivery blade 10 can be manufactured using well-known manufacturing techniques, such as, for example and without limitation, computer numerical control (“CNC”) machining. It is further contemplated that the drug delivery blade 10 can be formed of any suitable biocompatible material, such as, for example and without limitation, stainless steel.

When in use, the drug delivery blade of the invention is typically mounted on a blade holder. It is contemplated that the blade holder can provide a handle for holding and manipulating the drug delivery blade 10 and also provide a mechanism for advancing a plunger longitudinally through the lumen 14 of the delivery blade in order to eject a drug depot from the distal end of the lumen. In many embodiments, the blade holder can ergonomically configured for easy gripping and manipulation, and can generally have an overall shape similar to a pen or other writing instrument. It is contemplated that various mechanisms for advancing the plunger can be used. In some embodiments, the plunger can be advanced by pressing on the plunger at the distal end of the device, much like a conventional syringe would be operated. In other embodiments, the plunger can be advanced by applying a force normal to the housing of the blade holder. In these embodiments, an internal mechanism functions to transmit the normal force in order to advance the plunger in a longitudinal direction and eject the drug depot.

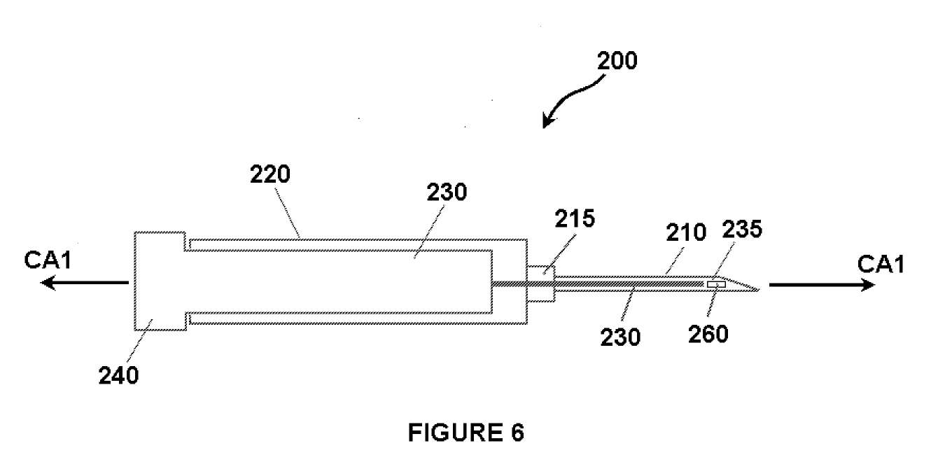

Referring now to FIG. 6, a blade holder 200 is shown attached to a drug delivery blade 210, such as described herein with respect to delivery blade 10. In an exemplary embodiment, blade holder 200 includes housing 220, which is configured to allow the delivery blade 210 to be held and manipulated by a surgeon. In this embodiment, the drug delivery blade 210 can be mounted on a distal end of holder 200. As shown in FIG. 6, the blade holder 200 and the drug delivery blade 210 can be aligned so as to have a common longitudinal axis CA1. It is contemplated that the mounting may be by a luer lock fitting 215, or other suitable attachment mechanism. On the inside, blade holder 200 can contain a plunger 230 that is appropriately sized and shaped to slide longitudinally through a lumen 235 of the delivery blade 210. In the embodiment of FIG. 6, the plunger 230 can be advanced by applying a force to the plunger 230 at a proximal end 240 of blade holder 200. As shown in FIG. 6, a drug depot 260 can initially be held in the lumen 235 of delivery blade 210 distal to the plunger 230. Upon application of force to the plunger 230, the plunger can be advanced along the common longitudinal axis CA1 through the lumen 235 toward a distal end of the lumen until causing the drug depot 260 to be ejected from the lumen.

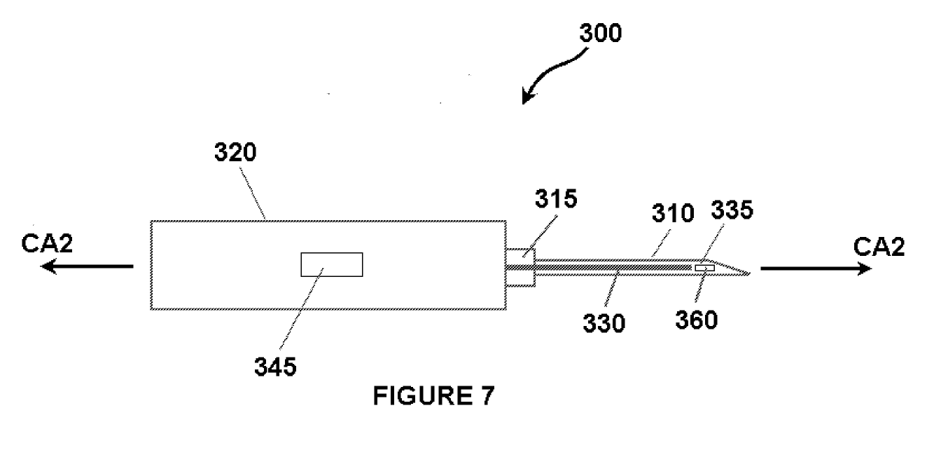

Referring now to FIG. 7, another embodiment of a blade holder 300 is shown attached to a drug delivery blade 310, such as described herein with respect to drug delivery blade 10. In an exemplary embodiment, the blade holder 300 can include a housing 320 that is configured to allow the delivery blade 310 to be held and manipulated by a surgeon. In this embodiment, the drug delivery blade 310 can be mounted on a distal end of holder 300. As shown in FIG. 7, the blade holder 300 and the drug delivery blade 310 can be aligned so as to have a common longitudinal axis CA2. It is contemplated that the mounting may be by a luer lock fitting 315, or other suitable attachment mechanism. On the inside, the blade holder 300 can contain a plunger 330 that is appropriately sized and shaped to slide longitudinally through a lumen 335 of the delivery blade 310. In the embodiment of FIG. 7, the plunger 330 can be advanced by applying pressure to the surface of a button 345 that is operatively coupled to the plunger 330 such that, upon application of pressure to the button 345, the plunger 330 is caused to be advanced along the common longitudinal axis CA1 through the lumen 335 toward the distal end of the lumen until causing the drug depot 360 to be ejected from the lumen. It is contemplated that any conventional electrical or mechanical means for operatively coupling the button 345 and the plunger 330 can be used as described herein. For example, it is contemplated that the button 345 can be in operative electrical communication with a controller that is configured to activate a motor placed in operative communication with the plunger 330 such that application of pressure to the button causes activation of the motor and, thus, a corresponding advancement of the plunger. Alternatively, it is contemplated that the button 345 can be mechanically coupled to the plunger 330 through a linkage (not shown) such that application of pressure to the button results in a corresponding, proportional advancement of the plunger. An exemplary device for causing selective advancement of a plunger is provided in U.S. Pat. No. 7,468,065, the disclosure of which is incorporated by reference herein in its entirety.

In various embodiments, the solid drug depots as described herein can have an overall length of about 10 mm or less. In an exemplary embodiment, the solid drug depot can have an overall length of about 7 mm or less. It is contemplated that drug depots having lengths of above 7 mm or more can interfere with a patient's vision after being placed in the vitreous of a patient. It is further contemplated that the drug depots can have a non-circular cross-sectional shape in order to approximately match the shape of the lumen of the drug delivery blade. It is still further contemplated that the drug delivery blades described herein can be used to deliver, for example and without limitation, implants, viscous polymers, in-situ gelling depots, microparticles, and the like.

The described drug delivery blade can be incorporated into methods of delivering a drug depot to a location in the eye of a patient. Various sites exist in the eye for implantation of a drug depot, such as, for example and without limitation, the vitreous of the eye, anterior or posterior chambers of the eye, or other areas of the eye including intraretinal, subretinal, intrachoroidal, suprachoroidal, intrascleral, episcleral, subconjunctival, intracorneal or epicorneal spaces. In one embodiment, a method of delivering drug depot into a patient's eye is provided. In this embodiment, the drug delivery blade is used to cut through an outer layer of a patient's eye with the blade, and the blade is then inserted and positioned at a desired location within the patient's eye or is otherwise advanced to a desired location in the patient's eye. In an exemplary embodiment, the drug delivery blade can be coupled to a blade holder as described herein. In this embodiment, once the shaft of the blade is positioned in the desired location, the plunger of the blade holder can be moved from the proximal end of the shaft toward the distal end of the shaft, thereby ejecting the drug depot from the lumen. After ejection, the blade can be removed from the patient's eye. It is contemplated that the incision created by the drug delivery blade can advantageously be self-sealing upon the removal of the blade from the eye. It is further contemplated that the self-sealing delivery, in addition to being less invasive and traumatic, can offer a less costly treatment by obviating the need for performing the procedure in a surgical setting.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A drug delivery blade comprising:
a shaft defining a longitudinal axis of the drug delivery blade, the shaft having a proximal end and a distal end;
a lumen that extends longitudinally from the proximal end of the shaft to the distal end of the shaft; wherein the lumen terminates at the distal end of the shaft to form an opening;
a first beveled cut that forms a sharpened tip at the distal end of the shaft; and
a flared portion proximate the distal end of the shaft, the flared portion comprising two radially extending blade segments,
wherein the radially-extending blade segments comprise sharpened leading edges at the distal end of the shaft.

2. The drug delivery blade of claim 1, wherein the first beveled cut is upwardly sloped angled relative to the longitudinal axis of the shaft at an angle ranging from about 15 degrees to about 45 degrees, as measured from the sharpened tip of the blade.

3. The drug delivery blade of claim 1, wherein the radially-extending blade segments are radially positioned at about 180 degrees relative to each other.

4. The drug delivery blade of claim 1, wherein the radially-extending blade segments each contain a sharpened leading edge that extends from the sharpened tip to an apex of each respective blade segment.

5. The drug delivery blade of claim 1, wherein the lumen has a non-circular cross-sectional shape.

6. The drug delivery blade of claim 1, wherein the lumen has an oval cross-sectional shape.

7. The drug delivery blade of claim 1, wherein the lumen has a cross-section shape comprising a square with rounded corners.

8. The drug delivery blade of claim 1, wherein the lumen comprises:
a substantially flat bottom surface;
substantially flat sidewall surfaces; and
a substantially arcuate top surface,
wherein the top and bottom surfaces meet the sidewall surfaces to define rounded corners.

9. The drug delivery blade of claim 1, wherein the lumen has a cross-sectional area ranging from about 0.1 mm² to about 0.4 mm².

10. The drug delivery blade of claim 1, wherein the lumen has a cross-sectional area ranging from about 0.1 mm² to about 0.2 mm².

11. The drug delivery blade of claim 1, wherein the drug delivery blade comprises one or more longitudinal bevel cuts on an underside of the blade.

12. The drug delivery blade of claim 11, wherein the longitudinal bevel cuts meet the first bevel cut to form the sharpened leading edges.

13. The drug delivery blade of claim 1, wherein the blade is configured to cut tissue as it is inserted into tissue but not cut tissue as the blade is withdrawn.

14. The drug delivery blade of claim 13, wherein each respective radially-extending blade segment comprises a trailing edge that is rounded such that the blade cuts tissue as it inserted into tissue but does not cut tissue as the blade is withdrawn.

15. The drug delivery blade of claim 1, wherein the drug delivery blade has a width ranging from about 0.5 mm to about 1.0 mm, as measured across the flared portion of the blade.

16. The drug delivery blade of claim 1, wherein the drug delivery blade has a width ranging from about 0.5 mm to about 0.8 mm.

17. The drug delivery blade of claim 1, wherein the drug delivery blade has a height ranging from about 0.3 mm to about 0.7 mm.

18. The drug delivery blade of claim 1, wherein the drug delivery blade has a height ranging from about 0.4 mm to about 0.6 mm.

19. The drug delivery blade of claim 1, wherein the sidewalls of the drug delivery blade have a thickness ranging from about 50 μm to about 100 μm.

20. The drug delivery blade of claim 1, wherein the drug delivery blade has a width of about 0.57 mm or less, a height of about 0.46 mm or less, and a sidewall thickness ranging from about 50 μm to about 60 μm.

21. The drug delivery blade of claim 1, wherein the drug delivery blade comprises stainless steel.

22. A drug delivery system comprising:
(a) a blade holder
(b) a drug delivery blade mounted thereon the blade holder, the drug delivery blade comprising:
a shaft defining a longitudinal axis of the drug delivery blade, the shaft having a proximal end and a distal end;
a lumen that extends longitudinally from the proximal end of the shaft to the distal end of the shaft; wherein the lumen terminates at the distal end of the shaft to form an opening;
a first beveled cut that forms a sharpened tip at the distal end of the shaft; and
a flared portion proximate the distal end of the shaft, wherein the flared portion comprises two radially extending blade segments,
wherein the radially-extending blade segments comprise sharpened leading edges at the distal end of the shaft; and
(c) a drug depot containing a drug,
wherein the blade holder comprises means for ejecting the drug depot from the opening of the drug delivery blade.

23. The drug delivery system of claim 22, wherein the drug depot comprises at least one of an implant, a viscous polymer, an in-situ gelling depot, and microparticles.

24. The drug delivery system of claim 22, wherein the drug depot has an overall length of about 10 mm or less.

25. The drug delivery system of claim 22, wherein the cross-sectional shape of the drug depot is configured to substantially match the cross-sectional shape of the lumen of the drug delivery blade.

26. A method of administering a drug to a target site in a patient, the method comprising the steps of:
(a) providing a drug delivery system comprising:
(i) a blade holder
(ii) a drug delivery blade mounted thereon the blade holder, the drug delivery blade comprising:
a shaft defining a longitudinal axis of the drug delivery blade, the shaft having a proximal end and a distal end;
a lumen that extends longitudinally from the proximal end of the shaft to the distal end of the shaft; wherein the lumen terminates at the distal end of the shaft to form an opening;
a first beveled cut that forms a sharpened tip at the distal end of the shaft; and
a flared portion proximate the distal end of the shaft, wherein the flared portion comprises two radially extending blade segments,
wherein the radially-extending blade segments comprise sharpened leading edges at the distal end of the shaft; and
(iii) a drug depot containing a drug,
wherein the blade holder comprises means for ejecting the drug depot from the opening of the drug delivery blade;
(b) creating an incision at the target site in the patient using the drug delivery blade; and
(c) implanting the drug depot at the target site using the blade holder to eject the drug depot from the opening of the lumen of the drug delivery blade.

27. The method of claim 26, wherein the target site is an eye of the patient.

28. The method of claim 27, wherein the target site is the vitreous of the eye of the patient.

29. The method of claim 26, wherein the incision is self-sealing.
